(19) United States
(12) Patent Application Publication
(10) Pub. No.: US 2014/0370103 A1
(43) Pub. Date: Dec. 18, 2014

(54) NON-IMMUNOGENIC DELIVERY VEHICLE FOR BETTA-, GAMMA-, AND DELTA-TOCOPHEROLS AND METHODS OF USING AND MAKING SAME

(71) Applicant: Phosphorex, Inc., Hopkington, MA (US)

(72) Inventor: Bin Wu, Sharon, MA (US)

(21) Appl. No.: 14/476,373

(22) Filed: Sep. 3, 2014

Related U.S. Application Data
(62) Division of application No. 13/443,538, filed on Apr. 10, 2012.

(51) Int. Cl. A61K 47/34; A61K 31/355; A61K 9/14

(52) U.S. Cl. CPC A61K 47/34; A61K 9/14; A61K 31/355
USPC 424/489; 514/458

(57) ABSTRACT

This disclosure provides microparticles for the delivery of non-immunogenic compositions providing delivery of β-, γ- and/or δ-tocopherols.

### BACKGROUND

Vitamin E (tocopherol) is a fat-soluble vitamin found in many vegetable seed oils and leafy green vegetables. Vitamin E has many functions including acting as an antioxidant of lipids, protection of cell membranes and prevention of damage to membrane associated enzymes. α-Tocopherol, often referred to as vitamin E, belongs to a class of lipid-soluble antioxidants that includes α, β, γ, and δ-tocopherols and α, β, γ, and δ-tocotrienols. Although α, β, γ, and δ-tocopherols and α, β, γ, and δ-tocopherols are sometimes referred to collectively as “vitamin E,”

α-Tocopherol is significant for human health, in part because it is readily absorbed and retained by the body, and therefore has a higher degree of bioactivity than other tocopherol species (Traber and Sies, Annu. Rev. Nutr. 16:321-347 (1996)). However, other tocopherols, such as β, γ, and δ-tocopherols, also have significant health and nutritional benefits.

α-Tocopherol is a major lipophilic antioxidant. It has been suggested that it plays an important role in preventing cardiovascular diseases and cancer. Due to increased interest in these protective as well as other beneficial effects, many vitamin E preparations have become widely available. Vitamin E is absorbed via the lymphatic systems where it is transported as lipoprotein complex. After oral administration, the vitamin is associated with mixed bile salt micelles in the small intestine to form a fine emulsion before moving across the epithelial cell membrane. It is then incorporated into a lipoprotein unit known as chylomicron or very low density lipoprotein before leaving the cell. These lipoprotein complexes, being too large to pass through the pores of the blood capillaries, are passed into the lymphatic vessels.

Recent advancement in cell biology showed how cells are damaged by certain reactive intermediates as well as how cellular processes are regulated by these reactive compounds in non-pathological conditions. The capacity of cells to “balance” redox states within relatively narrow limits established the boundary between reactive compounds that function as intracellular signaling factors and compounds that harm cells when produced in a manner that surpasses limits in their spatial and temporal frames of activity. This balance in redox state is in part controlled by the presence of other compounds that interact with the reactive intermediates associated with superoxide anion, nitric oxide, and hydrogen peroxide derived adducts. One of the prototypical modifiers is vitamin E, in particular the α-tocopherol isoform, which has received tremendous attention as an antioxidant in biological and chemical systems.

More recently, data have supported the concept that aberrant post translational chemical modification of proteins termed ‘nitrations’ significantly alter, and in most cases destroy, the biological activity of targeted proteins. Presently there are more than 70 disease states in humans that have a component of protein nitration associated with the problem, including acute respiratory disease stress (ARDS), arthrosclerosis, neurodegenerative disorders, and inflammatory immune diseases. Free radicals participate in these nitration-associated disease processes may not be interacting with the target proteins themselves, but rather through the activity of a highly reactive anion called peroxynitrite (ONOO-), which is formed by the aberrant interaction of two free radicals, nitric oxide and superoxide anion.

It has been recently observed in in vivo animal models that the impact of nitration stress of proinflammatory stress can be reduced by using subcutaneous injections of natural vitamin E, and that other isoforms such as γ-tocopherol and δ-tocopherol are more effective in reducing the nitration stress.

The fundamental chemistry of tocopherols suggests that while the α isoform may have beneficial effects in overall antioxidant capacity, it is the γ and δ isoforms that have few epitopes on the phenolic ring and indeed data indicate that both the γ and δ isoforms can directly adsorb ONOO- basically eliminating its ability to further nitrate other targets. Because of the two methyl groups on the phenolic reactive sites on the a isoform this ability to interact with the ONOO- is not present.

U.S. Pat. No. 6,908,043, incorporated herein by reference in its entirety, disclosed the use of y-tocopherol and its derivatives as antioxidants and nitrogen oxide scavengers which treat and prevent high blood pressure, thromboembolic disease, cardiovascular disease, cancer, natriuretic disease, the formation of neuropathological lesions, and a reduced immune system response.

While α-tocopherol is made available to tissues in vitro via its tocopherol binding protein, no such transport protein has been identified for the γ or δ isoforms. As such, the bioavailability of γ- and δ- is variable and short in duration. Furthermore while many biological attributes of the a isoform are capable of being explored using in vitro cell culture systems by using the water soluble acetate and succinate forms, no preparations of water soluble forms of γ- or δ- are available.

Vehicles for the delivery of a-tocopherol have been described, each of which is incorporated by reference in its entirety.) When a-tocopherol was replaced with γ- or δ-tocopherol in the microemulsion formulations provided by the prior art, no applicable and effective formula was produced. Therefore it is the intention of the current disclosure to develop a delivery vehicle for β-, γ-, and δ-tocopherols with high bioavailability.

### SUMMARY

This disclosure provides compositions and methods to increase the solubility and bioavailability of tocopherols. Said tocopherols include but are not limited to β-, γ-, and δ-tocopherols. In certain embodiments, any of the compositions described herein can be formulated to be non-immunogenic. In other embodiments, any of the compositions described herein can be formulated to be immunogenic.

The disclosure also provides a composition including a microemulsion comprising one or more polyglycol esters, one or more of β, γ or δ-tocopherol or mixtures thereof and water, wherein the microemulsion comprises droplets that are between 1 nm and 1000 nm in diameter. In certain embodiments, the droplets are between 10 and 200 nm in diameter. In certain embodiments, the composition is non-immunogenic. In other embodiments, the tocopherol can be any one of β, γ or δ-tocopherol or mixtures thereof. In other embodiments, the tocopherol can be y and/or 6-tocopherol or mixtures thereof.

In certain embodiments, the one or more polyglycol esters comprise a polyethylene glycol derivative of a mono-. di- or triglycerides of monofatty acides, and the mixture thereof. The monofatty acid can be selected from monolauric acid and mono caprylic acid. Specifically, the polyglycol ester can be a polyethylene glycol derivative of a mono-. di- or triglycerides of caprylic/capric acid, or the mixture thereof. The polyglycol ester can be triglycerol monolaurate, triglycerol monocaproate or triglycerol monocaprylate. More specifically, the polyglycol ester can be a PEG-8 caprylic/capric glyceride. One example of a PEG-8 caprylic/capric glyceride is Labrasol® a commercial product from Gattefossé.

In another embodiment, the non-immunogenic composition further includes an emulsifier. The emulsifier can be selected from ethanol, propylene glycol, diethylene glycol monoethyl ether, polyethylene glycols, polyglycerols, monoglycerides and lecithin. The emulsifier can also be selected from diethylene glycol monoethyl ether, monoglycerides and lecithin. Specifically, the emulsifier can be lecithin. In another embodiment, the non-immunogenic composition can also include a co-emulsifier. The co-emulsifier can be selected from ethanol, propylene glycol, diethylene glycol monoethyl ether, polyethylene glycols, polyglycerols, monoglycerides and lecithin. The co-emulsifier can also be selected from diethylene glycol monoethyl ether, monoglycerides and lecithin. Specifically, the co-emulsifier can be lecithin.

In another embodiment, the non-immunogenic composition also includes a carrier oil. The carrier oil can be selected from orange oil, palm oil, a triglyceride, squalane, squalene, limonene, isopropyl myristate and isopropyl palmitate. The carrier oil can also be selected from orange oil, squalane and limonene. Specifically, the carrier oil can be orange oil.

In other embodiments, the microemulsion includes between 1% and 20% or 0.1% and 99% by weight of tocopherol. The microemulsion can also include between 0.1% to 99% by weight of a polyglycol ester. The microemulsion can also include between 10% and 99% by weight of emulsifier. The microemulsion can also include between 0.1% and 89% by weight of co-emulsifier. In one embodiment, the ratio of emulsifier to co-emulsifier is between 40:60 and 60:40.

The disclosure provides a non-immunogenic composition comprising a microparticle between 1 nm and 1000 μm in diameter or between 20 nm and 500 nm in diameter comprising one or more of β, γ or δ-tocopherol and one or more polymers wherein the one or more polymers release the tocopherol upon administration to a subject. In certain embodiments the tocopherol can be any one of β, γ or δ-tocopherol or mixtures thereof. In other embodiments the tocopherol can be γ and/or δ-tocopherol or mixtures thereof.

In other embodiments, the polymer is biodegradable. The polymer can be selected from polylactic acid, poly(lactide-co-glycolide), polycaprolactone, polglycolic acid, polycaprolactone, poly(ethylene glycol), polyorthoesters, polyanhydrides, polycyanoacrylates, chitosan, dextran, and copolymers or combinations thereof, among others. Specifically, the polymer can be poly(D-L-lactide-co-glycolide) (PLGA). In a specific embodiment, the ratio of lactide to glycolide is between 100:0 and 0:100 weight percent. In another specific embodiment, the inherent viscosity of PLGA polymers is between 0.01 and 1.2 dL/g.

In another embodiment, the size of said microparticles is between 1 nm and 1,000 μm, between 10 nm and 100 μm or between 20 nm and 10 μm. In another embodiment, ratio of amount of the tocopherols to that of the polymer is between 1:1,000,000 and 100:1 or between 1:100 and 10:1.

In another embodiment, the non-immunogenic composition also includes a drug.

The disclosure also provides a method of making a drug delivery vehicle including the steps of dissolving a polymer in an organic solvent to form a solution; mixing one or more of β, γ or δ-tocopherol with the solution to form an emulsion; and mixing the emulsion with aqueous solution comprising a surfactant to form microparticles, thereby making a drug delivery vehicle. In certain embodiments the tocopherol can be any one of β, γ or δ-tocopherol. In other embodiments the tocopherol can be γ and/or δ-tocopherol. Said surfactant can be cationic, anionic, non-ionic or zwitterionic. Examples of surfactants include polyvinyl alcohol, polyvinyl acetate, polyvinylpyrrolidone, polyacrylic acid, Tween 20, Tween 80, Poloxamer 188, among others.

In other embodiments, the ratio of lactide to glycolide is between 100:0 and 0:100 weight percent. In other embodiments, the inherent viscosity of PLGA polymers is between 0.01 and 1.2 dL/g.

In certain embodiments, the size of said microparticles is between 1 nm and 1,000 μm, between 10 nm and 100 μm or between 20 nm and 10 μm. In other embodiments, the ratio of amount of the tocopherols to that of the PLGA is between 1:1,000,000 and 100:1 or between 1:100 and 10:1.

In another embodiment, the method also includes adding a drug to the emulsion. The disclosure also provides a foodstuff including a composition as defined above. In one embodiment, the foodstuff is selected from the group consisting of beverages, baked goods, cereals and dressing.

The disclosure also provides a pharmaceutical composition including a composition as defined above. In one embodiment, the pharmaceutical composition also includes a pharmaceutically acceptable excipient. The disclosure also provides methods of administering a tocopherol to an animal subject, by administering the pharmaceutical composition described above. The disclosure also provides a method of administering a drug to an animal subject by administering a composition or dosage form comprising a drug as described herein. The animal can be a human or non-human animal. Non-human animals include non-human primates, rodents, pets or livestock. Non-human primates include monkeys and apes. Rodents include mice, rats, hamsters, gerbils and rabbits. Pets include dogs, cats, birds and fish. Livestock include horses, cattle, goats, sheep, and pigs. The disclosure also provides a cosmetic material including a composition as defined above.

### DETAILED DESCRIPTION

This disclosure provides compositions and methods to increase the solubility and bioavailability of tocopherols. Said tocopherols include but are not limited to β-, γ-, and δ-tocopherols. One of the methods provided by the current disclosure is to encapsulate said tocopherols into small particles, i.e. microparticles or nanoparticles. The size of said microparticles and nanoparticles can be from 1 nanometer to 1,000 microns, from 10 nanometers to 100 microns, or from 20 nanometers to 10 microns. The ratio of amount of the tocopherols to that of the particles materials can be from 1:1,000,000 to 100:1, or 1:100 to 10:1.

Materials that can be used to encapsulate said tocopherols and form said particles can be synthetic polymers or polymers of natural origin. In one embodiment, the polymer used to encapsulate tocopherols is a biocompatible polymer that is capable of releasing the tocopherol to its site upon administration. In certain embodiments, the polymer is a biocompatible and biodegradable polymer. The polymer can be selected from polylactic acid, poly(lactide-co-glycolide), polycaprolactone, polglycolic acid, polycaprolactone, poly(ethylene glycol), polyorthoesters, polyanhydrides, polycyanoacrylates, chitosan, dextran, and copolymers or combinations thereof, among others. One aspect of the disclosure is a composition including poly(D-L-lactide-co-glycolide) (PLGA) particles encapsulating a tocopherol, wherein the ratio of lactide to glycolide is from about 100:0 to about 0:100 weight percent; the inherent viscosity of PLGA polymers used in the particles is from about 0.01 to about 1.2 dL/g.

Another aspect of the disclosure is a composition for use as a drug delivery vehicle including tocopherols encapsulated in PLGA particles. Another aspect of the disclosure is a method for encapsulating tocopherols in microparticles, including (a) dissolving PLGA polymer in an organic solvent to produce a solution; (b) adding tocopherol to the solution of (a) to produce a PLGA-tocopherol emulsion; (c) adding a large amount of aqueous solution containing a surfactant to harden the microparticles . in certain other embodiments, a double emulsion/solvent evaporation technique can be used to form the microparticles. Particle formation systems are described in U.S. Pat. No. 3,523,907, Ogawa et al., Chem Plarm. Bull. (1988) 36:1095-1103, O'Hagan et al., Vaccine (1993) 11:965-969, PCT/US99/17308 (WO 00/06123) to O'Hagan et al. and Jeffery et al., Pharm. Res. (1993) 10:362, incorporated herein by reference in their entireties.

Microparticle compositions may be produced, for example, by a method that comprises (a) forming a water-in-oil emulsion by emulsifying an organic liquid like that above and an immiscible aqueous liquid. like that above; (b) forming a water-in-oil-in-water emulsion by emulsifying (1) the thus-formed water-in-oil emulsion with (ii) an additional aqueous liquid comprising water (and which may optionally comprise a surfactant); and (c) removing the organic solvent from the water-in-oil-in-water emulsion to form microparticles,

Another method provided in this disclosure is to create a microemulsion including said tocopherols and aqueous media.

A microemulsion is a thermodynamically stable isotropically clear dispersion of two immiscible liquids, such as oil and water, stabilized by an interfacial film of surfactant molecules. In certain embodiments, the microemulsion described herein has a mean droplet diameter of less than 200 nm, or between 10-50 nm. In the absence of water, mixtures of oil(s) and non-ionic surfactant(s) form clear and isotropic solutions that are known as self-emulsifying drug delivery systems (SEDDS) and have successfully been used to improve lipophilic drug dissolution and oral absorption. In certain embodiments, SEDDS are used to deliver tocopherols according to the instant disclosure.

Part of the present disclosure is directed to compositions of tocopherols in the form of emulsions, micellar solutions or self-emulsifying drug delivery systems. The compositions of the disclosure contain one or more of β, γ, or δ-tocopherol and can be administered to animals or humans via intravascular, oral, intramuscular, cutaneous and subcutaneous routes. Non-human animals include non-human primates, rodents, pets or livestock. Non-human primates include monkeys and apes. Rodents include mice, rats, hamsters, gerbils and rabbits. Pets include dogs, cats, birds and fish. Livestock include horses, cattle, goats, sheep, and pigs. Specifically, the microemulsions can be given by any of the following routes, among others: intraabdominal, intraarterial, intraarticular, intracapsular, intracervical, intracranial, intraductal, intradural, intralesional, intralocular, intralumbar, intramural, intraocular, intraoperative, intraparietal, intraperitoneal, intrapleural, intrapulmonary, intraspinal, intrathoracic, intratracheal, intratympanic, intrauterine, and intraventricular. The emulsions described herein can also be nebulized using suitable aerosol propellants which are known in the art for pulmonary delivery of lipophilic compounds.

The microemulsion can include as an emulsifier, at least one polyglycol ester. In certain embodiments, the one or more polyglycol esters comprise a polyethylene glycol derivative of a mono-. di- or triglycerides of monofatty acides, and the mixture thereof. The monofatty acid can be selected from monolauric acid and mono caprylic acid. Specifically, the polyglycol ester can be a polyethylene glycol derivative of a mono-. di- or triglycerides of caprylic/capric acid, or the mixture thereof. More specifically, the polyglycol ester can be a PEG-8 caprylic/capric glyceride. One example of a PEG-8 caprylic/capric glyceride is Labrasol®, a commercial product from Gattefossé S.A., France.

With this microemulsion it is possible to solubilize the aforementioned tocopherols in any amount of water without changing the microscopic appearance of the microemulsion or without breaking the microemulsion.

In certain embodiments, the emulsifiers used are non-toxic and, accordingly, such microemulsions can be used in foodstuffs or the pharmaceutical or cosmetic field.

The microemulsions can be manufactured with or without a co-emulsifier. Co-emulsifiers include ethanol, propylene glycol, Transcutol® (diethylene glycol monoethyl ether, available from Gattefossé S.A., France), polyethylene glycols, polyglycerols, monoglycerides or lecithin, especially Transcutol®, monoglycerides or lecithin, with lecithin being especially preferred.

Furthermore, the microemulsion can also contain at least one carrier oil. The tocopherol is dissolved at least in part in the carrier oil, which increases the final content of lipophilic substance in the finished microemulsion. The carrier oil can be orange oil, palm oil, a triglyceride, squalane, squalene, limonene, isopropyl myristate or isopropyl palmitate. In certain embodiments, the carrier oil is orange oil, squalane or limonene. In one embodiment, the carrier oil is orange oil.

The microemulsion can also contain a carotenoid. Carotenoids include all-trans-β-carotene, 9-cis-β-carotene, 13-cis-β-carotene, β-opo-8′-carotenic acid ethyl ester, apocarotenal, astaxanthin, canthaxanthin, crocetin or lycopene. The carotenoid can also be β-carotene.

In certain embodiments of the microemulsion, the amount of tocopherol is between 0.1 and 99 wt. %, the amount of emulsifier is between 10 and 99 wt. % and the amount of co-emulsifier is between at 0 and 89 wt. %, with the ratio of emulsifier to co-emulsifier being between 40:60 and 60:40. The ratio of emulsifier to co-emulsifier can be about 50:50 wt. %.

For foodstuffs, the oil in water microemulsions of the present disclosure provides a convenient way to add or fortify a variety of foodstuffs, for examples, beverages, baked goods, dressing, and the like, with the lipophilic substances mentioned herein. The same holds true for pharmaceutical compositions and cosmetics. For pharmaceutical compositions, the oil in water microemulsions permit the addition of the lipophilic substance to pharmaceutical compositions, for example, oral vitamin solutions. For cosmetics, the oil in water microemulsions permit the addition of the lipophilic substances to variety of cosmetics, for example, foundation, lipstick, and the like.

In each instance for foodstuff, pharmaceutical, and cosmetic materials, the addition of the lipophilic substances mentioned herein provide valuable properties to the materials as one of ordinary skill in the art will appreciate when such lipophilic substances are either consumed (foodstuffs or pharmaceuticals) or are topically applied (pharmaceuticals or cosmetics). For the various foodstuff, pharmaceutical and cosmetic materials, the amounts of oil in water microemulsion to be added will depend upon the amount of lipophilic substance that is desired to be added to the material and the effect which the lipophilic substance is to have, for example, antioxidant effect or taking in the recommended daily allowance of the lipophilic substances.

In the case of foodstuffs, by fortifying the foodstuffs with the oil in water microemulsion of the present application, the lipophilic substance incorporated by use of the microemulsion provides for levels of the lipophilic substance which would be greater than that present in the foodstuff without the addition of the microemulsion. In the case of cosmetics, by adding the microemulsion described herein, the cosmetics can be fortified with many of the lipophilic substances which are considered useful in treating wrinkles, burns, etc. In the case of pharmaceuticals, using the microemulsions described herein permits valuable formulations to be fortified with, for example, the lipophilic substances mentioned herein to make vitamin solutions and other medicaments to be prepared.

The microemulsion is dilutable or miscible with water in any ratio. A pharmaceutically convenient dilution of the microemulsion contains e.g. between 80 and 95 wt % water. In certain embodiments, the microemulsion contains about 90 wt. % water. In these formulations 0.1-2 wt. % tocopherol can be present.

The speed of the microemulsion formation depends on the velocity at which the individual components dissolve. When salve-like emulsifiers, such as, triglycerol monolaurate are used, the speed at which these emulsifiers dissolve can be accelerated by slight stirring and possibly warming to about 40-45° C. A magnetic stirrer is suitable for this purpose. However, a different stirrer or any heating system can be used. The formulation is finished when a clear, isotropic liquid has formed from the individual components, which usually occurs after several minutes.

The formulations can additionally contain flavorants, colorants and/or thickeners when their use requires this to be the case. If the content of polyglycerol esters amounts to less than about 3-5%, the formulation can be additionally preserved with conventional preservatives.

The tocopherol microemulsion is also suitable as delivery vehicle for poorly soluble drugs. Poorly soluble drugs may be dissolved in tocopherols and the resulting oily drug solution can be further incorporated into a microemulsion, thus facilitating the delivery of the poorly soluble drug.

Definitions As used in this specification and any appended claims, the singular forms “a,” “an” and “the” include plural references unless the content clearly dictates otherwise. Thus, for example, the term “microparticle” refers to one or more microparticles, and the like.

Unless stated otherwise or unless the context clearly dictates otherwise, all percentages and ratios herein are given on a weight basis.

The term “microparticle” as used herein, refers to a particle of less than 1000 micrometers (μm or microns) in diameter, including nanoparticles.

The term “nanoparticle” as used herein, refers to a particle of less than 1,000 nm in diameter.

Particle size can be determined or measured using methods available in the art. For example, particle size can be determined using photon correlation spectroscopy, dynamic light scattering or quasi-elastic light scattering. Particle size can also be determined using static light scattering, which measures the intensity of light scattered by particles in a solution at a single time. In certain embodiments, particle size is determined at room temperature and involves multiple analyses of the sample in question (e.g., at least 3 repeat measurements on the same sample) to yield an average value for the particle diameter.

The microparticles according to the present disclosure may vary in size. Microparticles can range in diameter from 1 nanometer (nm) to 1,000 microns. In other embodiments, microparticles range in diameter from 10 nm to 100 microns. In other embodiments, microparticles range in diameter from 20 nm to 10 microns, from 100 nm to 50 microns, from 500 nm to 25 microns, and from 1 micron to 10 microns. In certain embodiments, according to the present disclosure microparticles have diameters of 1, 10, 100, 200, 300, 400, 500, 600, 700, 800, 900 or 1000 nm. In other embodiments, according to the present disclosure microparticles have diameters of 1, 2, 5, 10, 20, 50, 100, 200, 300, 400, 500, 600, 700, 800, 900 or 1000.

As defined herein, an “aqueous liquid” is a water-containing liquid, typically a liquid containing more than 50 wt % water. In certain embodiment an aqueous liquid contains from 50 to 75 wt % water or from 90 to 95 wt % water. In other embodiments, an aqueous liquid contains 50, 60, 70, 80, 90, 95, 96, 97, 98, 99 or 100 wt % water. As defined herein, an “aqueous solvent” is a water-containing solvent, typically a solvent containing more than 50 wt % water. In certain embodiment an aqueous solvent contains from 50 to 75 wt % water or from 90 to 95 wt % water. In other embodiments, an aqueous solvent contains 50, 60, 70, 80, 90, 95, 96, 97, 98, 99 or 100 wt % water.

As defined herein, an “organic liquid” is a liquid that contains one or more organic solvent species, typically a liquid containing from 50 to 75 wt % organic solvent species or from 90 to 95 wt % organic solvent species. In other embodiments, an organic liquid contains 50, 60, 70, 80, 90, 95, 96, 97, 98, 99 or 100 wt % organic solvent species.

As defined herein, an “organic solvent” is a solvent containing one or more organic solvent species, typically a solvent containing from 50 to 75 wt % organic solvent species or from 90 to 95 wt % organic solvent species. In other embodiments, an organic solvent contains 50, 60, 70, 80, 90, 95, 96, 97, 98, 99 or 100 wt % organic solvent species.

As defined herein, an “organic solvent species” is a liquid, solid, or gas that dissolves another solid, liquid, or gaseous solute, resulting in a solution that is soluble in a certain volume of solvent at a specified temperature.

As defined herein, a “microparticle suspension” is a liquid phase that contains microparticles.

As defined herein, an “aqueous microparticle suspension” is an aqueous liquid that further contains microparticles.

The term “surfactant,” as used herein, refers to substances that accumulate at interfaces (e.g., at liquid-liquid, liquid-solid and/or liquid-gas interfaces) and change the properties of that interface. As used herein, surfactants include detergents, dispersing agents, suspending agents, emulsion stabilizers, and the like. Said surfactant can be cationic, anionic, non-ionic or zwitterionic. Preferred surfactants include polyvinyl alcohol, polyvinyl acetate, polyvinylpyrrolidone, polyacrylic acid, sodium dodecylsulfate (SDS), Tween 20, Tween 80, Poloxamer 188, among others. The term “pharmaceutical,” as used herein, refers to biologically active compounds such as antibiotics, antiviral agents, growth factors, hormones, and the like. The term “drug” is synonymous with pharmaceutical.

The term “adjuvant,” as used herein, refers to any substance that assists or modifies the action of a pharmaceutical, including but not limited to immunological adjuvants, which increase and/or diversify the immune response to an antigen. Hence, immunological adjuvants are compounds that are capable of potentiating an immune response to antigens. Immunological adjuvants can potentiate humoral and/or cellular immunity. In some embodiments, immunological adjuvants stimulate an innate immune response. Immunological adjuvants may also be referred to as “immunopotentiators.” Adjuvants are further described in U.S. Publication No. 2011/0280949, incorporated herein by reference in its entirety.

As used herein, an “antigen” refers to a molecule containing one or more epitopes (e.g., linear, conformational or both) that elicit an immunological response. The term may be used interchangeably with the term “immunogen.” By “elicit” is meant to induce, promote, enhance or modulate an immune response or immune reaction. In some instances, the immune response or immune reaction is a humoral and/or cellular response. An antigen may induce, promote, enhance or modulate an immune response or immune reaction in cells in vitro and/or in vivo in a subject and/or ex vivo in a subject's cells or tissues. Such immune response or reaction may include, but is not limited to, eliciting the formation of antibodies in a subject, or generating a specific population of lymphocytes reactive with the antigen. Antigens are typically macromolecules (e.g., proteins, polysaccharides, polynucleotides) that are foreign to the host. Antigens are further described in U.S. Publication No. 2011/0280949, incorporated herein by reference in its entirety.

As used herein, an “epitope” is that portion of given species (e.g., an antigenic molecule or antigenic complex) that determines its immunological specificity. An epitope is within the scope of the present definition of antigen. Epitopes are further described in U.S. Publication No. 2011/0280949, incorporated herein by reference in its entirety.

An “immunological response” or “immune response” to an antigen or composition is the development in a subject of a humoral and/or a cellular immune response to molecules present in the composition of interest. Immunological or immune responses are further described in U.S. Publication No. 2011/0280949, incorporated herein by reference in its entirety.

An “immunogenic composition,” as defined herein, provides “enhanced immunogenicity” for a given antigen when they possess a greater capacity to elicit an immune response than the immune response elicited by an equivalent amount of the antigen in a differing composition (e.g., wherein the antigen is administered as a soluble protein). Thus, a composition may display “enhanced immunogenicity,” for example, because the composition generates a stronger immune response, or because a lower dose or fewer doses of antigen is necessary to achieve an immune response in the subject to which it is administered. Such enhanced immunogenicity can be determined, for example, by administering the compositions described herein, and antigen controls, to animals and comparing assay results of the two. Immunogenic compositions are further described in U.S. Publication No. 2011/0280949, incorporated herein by reference in its entirety.

The term “non-immunogenic” as defined herein, refers to a composition that does not provide enhanced immunogenicity. In certain embodiments, a non-immunogenic composition comprises no adjuvant. In other embodiments, a non-immunogenic composition comprises less than 5% adjuvant by weight. In other embodiments, a non-immunogenic composition comprises less than 4, 3, 2, 1, 0.1, 0.05, 0.01, 0.001, 0.0005 or 0.00001% adjuvant by weight. In other embodiments, a non-immunogenic compound does not include an antigen with an epitope that elicits an immune response. In other embodiments, a non-immunogenic compound comprises less than 4, 3, 2, 1, 0.1, 0.05, 0.01, 0.001, 0.0005 or 0.00001% of an antigen with an epitope that elicits an immune response by weight.

As used herein, “treatment” (including variations thereof, for example, “treat” or “treated”) refers to any of (i) the prevention of a pathogen or disorder in question (e.g. cancer or a pathogenic infection, as in a traditional vaccine), (ii) the reduction or elimination of symptoms associated with a pathogen or disorder in question, and (iii) the substantial or complete elimination of a pathogen or disorder in question. Treatment may thus be effected prophylactically (prior to arrival of the pathogen or disorder in question) or therapeutically (following arrival of the same).

The terms “effective amount” or “pharmaceutically effective amount” of a composition of the present disclosure refer herein to a sufficient amount of the composition to treat or diagnose a condition of interest. The exact amount required will vary from subject to subject, depending, for example, on the species, age, and general condition of the subject; the severity of the condition being treated. An appropriate “effective” amount in any individual case may be determined by one of ordinary skill in the art. Thus, a “therapeutically effective amount” will typically fall in a relatively broad range that can be determined through routine trials.

By “subject” is meant any animal, including, without limitation, mammals such as cattle, sheep, pigs, goats, horses, rats, mice, rabbits and humans; domestic animals such as dogs and cats; and birds, including domestic, wild and game birds such as cocks and hens including chickens, turkeys and other gallinaceous birds. The term does not denote a particular age. Thus, both adult and newborn animals are covered.

By “pharmaceutically acceptable” or “pharmacologically acceptable” is meant a material which is not biologically or otherwise undesirable, e.g., the material may be administered to an individual without causing any excessively undesirable biological effects in the individual or interacting in an excessively deleterious manner with any of the components of the composition in which it is contained.

The term “excipient” refers to any essentially accessory substance that may be present in the finished dosage form. For example, the term “excipient” includes vehicles, binders, disintegrants, fillers (diluents), lubricants, suspending/dispersing agents, and so forth.

As used herein, “biodegradable” refers to a substance that can be metabolized by living things. These living things include bacteria, fungi, plants, animals and protists.

As used herein, “release” upon administration to a subject refers to the release from a dosage form or administered composition of greater than 50% of a given substance in the dosage form or administered composition. For example, a dosage form containing tocopherol that releases the tocopherol upon administration to a subject would release at least 50% of the tocopherol to the subject. This release tocopherol would be available for metabolism by the subject. This metabolism would include interaction of the tocopherol in redox reactions in the subject. In other embodiments, release can refer to 60, 70, 80, 90, 95, 96, 97, 98, 99 or 100% of a given substance in a dosage form or administered composition.

The general principles of the present disclosure may be more fully appreciated by reference to the following non-limiting examples.

### EXAMPLES

### Example 1

Encapsulation of δ-tocopherol in PLGA Nanospheres.

100 milligrams of PLGA having an L/G ratio of 50/50 and an inherent viscosity of 0.15-0.25 in chloroform was dissolved in 10 ml methylene chloride. 12 milligrams of 6-tocopherol was mixed with the PLGA solution. After the tocopherol was completely dissolved, the solution was mixed with 150 ml 1% polyvinyl alcohol solution in a 250 ml glass bottle. The mixture was transferred to a 300 ml container and homogenized at 15,000 rpm for 1.5 minutes, after which the emulsion was stirred magnetically for 4 hours and 10 minutes. The resulting suspension was centrifuged. After the supernatant was decanted, 1 ml 10% mannitol solution and 1 ml deionized water were added to the nanospheres pellet. After vortexing, the tocopherol nanospheres were lyophilized. Such obtained nanospheres were found to have a mean particle size of 423 nm by a laser diffraction particle size analyzer. The tocopherol amount encapsulated inside the nanospheres was found by a UV-Vis photospectrometer to be 7.4 w/w %.

### Example 2

### Microemulsion of δ-Tocopherol.

1 gram of 6-tocopherol was mixed with 5 grams of Labrasol® and 10 grams of deionized water in a 15 ml glass vial. The glass vial was rotated on a rotator for approximately 5 hours. A clear microemulsion liquid was obtained.

We claim:

1. A composition comprising a microemulsion comprising one or more polyglycol esters, one or more of β, γ or δ-tocopherol and water, wherein the microemulsion comprises droplets between 1 nm and 1000 nm in diameter.

2. The composition of claim 1, wherein the droplets are between 10 nm and 200 nm in diameter.

3. The composition of claim 1, wherein the composition is non-immunogenic.

4. The composition of claim 1 comprising β-tocopherol.

5. The composition of claim 1 comprising γ-tocopherol.

6. The composition of claim 1 comprising δ-tocopherol.

7. The composition of claim 1, wherein the one or more polyglycol esters is a polyethylene glycol derivative of a mono- di- or triglycerides of monofatty acids.

8. The composition of claim 1, wherein the one or more polyglycol esters comprise PEG-8 caprylic/capric glyceride.

9. The composition of claim 1 further comprising an emulsifier.

10. The composition of claim 9, wherein the emulsifier is selected from the group consisting of ethanol, propylene glycol, diethylene glycol monoethyl ether, polyethylene glycols, polyglycerols, monoglycerides and lecithin.

11. The composition of claim 1 further comprising a co-emulsifier.

12. The composition of claim 11, wherein the co-emulsifier is selected from the group consisting of ethanol, propylene glycol, diethylene glycol monoethyl ether, polyethylene glycols, polyglycerols, monoglycerides and lecithin.

13. The composition of claim 1 further comprising a carrier oil.

14. The composition of claim 13, wherein the carrier oil is selected from the group consisting of orange oil, palm oil, a triglyceride, squalane, squalene, limonene, isopropyl myristate and isopropyl palmitate.

15. The composition of claim 1, wherein the microemulsion comprises between 1% and 20% by weight of tocopherol.

16. The composition of claim 11, wherein the microemulsion comprises between 0.1% and 89% by weight of co-emulsifier.

17. A non-immunogenic composition comprising a microparticle between 20 nm and 500 nm in diameter comprising one or more of β, γ or δ-tocopherol and one or more polymers wherein the one or more polymers release the tocopherol upon administration to a subject.

18. The non-immunogenic composition of claim 17, wherein the polymer is biodegradable.

19. The non-immunogenic composition of claim 17, wherein the polymer is selected from the group consisting of poly(lactide-co-glycolide), poly(lactic acid), poly(glycolic acid), polyhydroxyalkanoates, polycaprolactones, poly(ethylene glycol), polyorthoesters, polyanhydrides, polycyanoacrylates, chitosan, dextran, and copolymers or combinations thereof.

20. The non-immunogenic composition of claim 17, wherein the polymer is poly(D-L-lactide-co-glycolide) (PLGA).

21. The non-immunogenic composition of claim 20, wherein the ratio of lactide to glycolide is between 100:0 and 0:100 weight percent.

22. The non-immunogenic composition of claim 17, further comprising a drug.

23. A method of making a drug delivery vehicle comprising
a) dissolving a polymer in an organic solvent to form a solution;
b) mixing one or more of β, γ or δ-tocopherol with the solution to form an emulsion; and
c) mixing the emulsion with aqueous solution comprising a surfactant to form microparticles,
thereby making a drug delivery vehicle.

24. A foodstuff comprising a composition according to claim 1.

25. A pharmaceutical composition comprising a composition according to claim 1.

26. A method of administering tocopherol to an animal subject comprising administering the pharmaceutical composition of claim 25.

27. A cosmetic material comprising a composition according to claim 1.
